(19) United States
(12) Patent Application Publication
(10) Pub. No.: US 2008/0033551 A1
(43) Pub. Date: Feb. 7, 2008

(54) Biocompatible surface modifications for metal orthopedic implants

(76) Inventor: John E. Kelley, Gaston, OR (US)

Correspondence Address: CHERNOFF, VILHAUER, MCCLUNG & STENZEL, 1600 ODS TOWER, 601 SW SECOND AVENUE, PORTLAND, OR, 97204-3157, US

(21) Appl. No.: 11/810,152

(22) Filed: Jun. 5, 2007

Related U.S. Application Data
(63) Continuation of application No. PCT/US04/40458, filed on Dec. 2, 2004.
(60) Provisional application No. 60/526,471, filed on Dec. 2, 2003.

(51) Int. Cl. A61F 2/28; A61K 33/42; A61L 27/32

(52) U.S. Cl. 623/16.11; 424/602; 427/2.27; 427/540; 623/23.51

(57) ABSTRACT

A biocompatible implant comprising a surface layer metallurgically bonded to a substrate and incorporating one or more tissue-growth enhancing materials such as calcium or phosphorus therein. The implant may formed by a submerged arc welding process, or other suitable methods.

This application claims priority to PCT/US2004/040458, filed Dec. 2, 2004, which claims priority to U.S. 60/526,471, filed Dec. 2, 2003.

### BACKGROUND

This invention relates to biocompatible implants, and in particular to implants that promote the growth and attachment of tissue to the implant Biocompatible implants are commonly used to secure or to replace bone structures in humans and animals. Implants used to maintain and extend the functionality of limbs, joints, and dental functions are typically made from corrosion resistant metal materials, such as stainless steels, cobalt-chromium molybdenum alloys, or titanium alloys. They are commonly applied to hips, knees, shoulders, hands, jaws, and other areas where stabilization may be required, such as vertebra segments or support rods for the backbone. In other applications implants are used to reinforce or reshape vascular structures such as aneurisms. Advancements in implant technology have included the development of coatings for implants that improve the ability of the body to accept the implant, as well as the ability to accelerate the growth and attachment of body tissues onto the implant. Typical approaches employed include the attachment to the implant surface of high surface area metal beads or high surface area hydroxyapatite (HA), which is the chemical equivalent of bone onto the implant. Typical approaches employed include the attachment to the implant surface of high surface area metal beads, or high surface area hydroxyapatite (HA), which is the chemical equivalent of bone. These surface coatings provide both chemical compatibility, as well as a textured surface onto which the body tissues can firmly attach.

While these advancements have reduced the rejection rate of implants in human and animal recipients, they also suffer from metallurgical property shortcomings that result in premature failure of the implant, rejection by the recipient, and/or damage to the surrounding bone and tissue in the recipient. There are several major shortcomings of current technologies.

In the case of high surface area metal surfaces, such as titanium spheres that are sintered onto the implant, the issue is that of tissue compatibility. Even if tissue grows into the porous structure provide by the coating, the bond between the tissue and the titanium coating is strictly mechanical rather than biological. Because the bone tissue sees the metal surfaces as a “foreign” material, de-bonding occurs over time, and the implant fails to perform according to design.

Another shortcoming of the prior art is that surface coatings of metal or HA are mechanically bonded to the underlying implant surface. Over time the surface coatings de-bond from the implant body. Debonding of the implant coatings causes mechanical failure of the implant and/or rejection of the implants.

Finally, most mechanically bonded metal and HA coatings applied today are the result of either thermal spray technology or a sintering process, both of which expose the base metal (or implant) to high temperatures. This exposure can result in the formation of a heat affected zone (HAZ) within the base metal or implant. An HAZ can result in premature fatigue cracking of the implant, as can compromise other important properties of the implant such as tensile strength and Young's Modulus.

In any of the above, the result is often the premature failure of the implant and premature replacement surgery, exposing the patient to the inherent risks, expense and inconvenience of additional surgery. Clearly, technological advances in this area that could improve the bond of surface layers to the body of the implant while at the same time enhancing the growth and attachment of tissue to the implant would represent a major improvement in implant technology.

### SUMMARY OF THE INVENTION

This invention provides improved biocompatible implants that exhibit improved structural integrity when compared to known implants, and that accelerate the growth and attachment of body tissues to the implant. The invention is embodied in implant devices that include an underlying structure and a surface layer deposited on the underlying structure by a method known as fusion surfacing.

Pulse fusion surfacing (PFS) refers to a pulsed-arc micro-welding process that uses short-duration, high current electrical pulses to deposit an electrode material onto a metallic substrate. PFS allows a fused, metallurgically bonded coating to be applied with a sufficiently low total heat output so that the bulk substrate material remains at or near ambient temperatures. The short duration of the electrical pulse allows an extremely rapid solidification of the deposited material and results in a fine-grained, homogeneous coating that approaches an amorphous structure. The process has been used in the past to apply wear and corrosion resistant surfaces on materials used in harsh environments. Alternative coatings have been used to alter the substrate surface resistance to wear and corrosion.

PFS is generally described in U.S. Pat. No. 5,448,035 to Thutt, Kelley et al., which is hereby incorporated by reference in its entirety. In general, PFS is a welding method in which very small, pulsed electrical currents are discharged through an electrode into a workpiece, in this instance an implant. The current pulses melt small portions of the electrode and at the same time heat and melt a very thin layer of a small portion of the surface. The molten electrode material is welded to the surface while the workpiece remains largely unaffected since the current pulses are so small. The result is a very thin layer of alloy “welded” to the surface of the workpiece. The alloy can be chosen to provide wear resistance, chemical resistance, surface hardness or any of a number of desired properties. In a PFS process both the electrode and the workpiece (i.e., substrate) are conductive and form the terminal poles of a direct current power source. When a high surge of energy is applied to the electrode, a spark is generated between the electrode and the substrate. While not known for sure, it is generally assumed that a gas bubble forms about the spark discharge from the electrode and persists for a time longer than the spark itself. Metal melted due to the high temperature of the spark is then transferred from the electrode to the substrate surface via the expanding gas bubble. Alternatively, the polarities between the electrode and the substrate can be reversed so that metal can be transferred from the substrate to the electrode.

The PFS surface layer as used in the present invention is formed of any of a number of metallic or ceramic alloys, or can be formed of the same material as the implant or workpiece. The PFS surface layer according to this invention includes one or more tissue growth-enhancing elements such as calcium or phosphorous integrated into the PFS-formed surface layer, and which stimulate tissue growth and attachment to the PFS-applied surface layer. The PFS layer of the present invention is applied by a novel method in which the underlying structure is immersed in a liquid bath containing one or more dissolved tissue growth enhancing elements. The PFS layer can be tailored in both composition and surface morphology to provide any number of properties as is described in the prior art. In addition, however, this invention provides a significant additional feature that has heretofore not been possible. In this invention the PFS layer is applied with the electrode and workpiece submerged in a liquid bath. The liquid bath contains one or more tissue-growth enhancing elements or compounds in solution or in suspension that are integrated into the PFS layer as it is applied to the workpiece. The tissue-growth enhancing elements promote the growth and attachment of tissue to the implant, leading to a more reliable and durable treatment when implants are required.

The invention is embodied in orthopedic implants such as hip and knee implants, spinal inserts, orthopedic and dental attachment devices such as screws and wires, cardiac devices, and vascular implants such as vascular occlusive devices used to treat aneurysms. This list is intended to be inclusive and not exhaustive.

### BRIEF DESCRIPTION OF THE DRAWINGS

FIG. 1 is a schematic view of a processing bath according to the invention.

### DESCRIPTION OF THE INVENTION

Preferred embodiments of the invention will now be described in greater detail by reference to the drawings and several examples.

### EXAMPLE 1

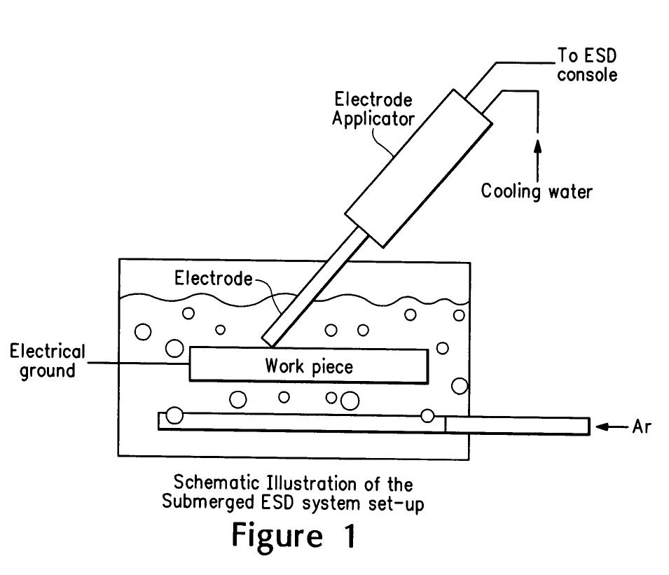

In one example, a liquid bath (FIG. 1) was made from a mixture of 69 grams of distilled water, 10 grams of calcium carbonate, and 82 grams of phosphoric acid (H3PO4), and 52 grams of calcium phosphate (monobasic monohydrate). A sample disc of Ti-6Al-4V was submerged in the bath, grounded to the PFS circuit, and supported by a non-conductive polymeric support. A stream of argon was bubbled into the bottom of the bath for agitation. A suitable PFS system is currently made and sold by Advanced Surfaces and Processes, Inc., assignee of the present invention. A PFS electrode of the same alloy was connected to the PFS apparatus, and placed in operative proximity to the sample. A relatively low energy PFS process was then conducted for about 3 minutes during which current was passed through the electrode and into the sample. The sample was then removed from the bath, ultrasonically cleaned, and analyzed by Energy-Dispersive X-Ray Spectroscopy (EDX) for calcium and phosphorous content. The PFS-applied layer included 0.34 atomic % calcium and 1.54 atomic % phosphorous. The sample was then tested for tissue-growth enhancement.

Primary rat osteoblasts were seeded onto the sterile surface of the sample and onto the sterile surface of an unmodified Ti-6Al-4V sample by placing each sample into a well containing 10,000 cells per disc in a 100 milliliter volume of tissue culture media (alpha MEM, supplemented with 5% FBS, (Gibco). Following a 1, 4 and 7 day culture period, attachment and proliferation was measured with the metabolic indicator Alamar Blue (Biosource International, Camarillo, Calif.). Alamar blue is a non-destructive oxidation-reduction calorimetric indicator that enables repeated analysis of each sample over several intervals. The cell culture medium was removed from each well and was replaced with a 100% Alamar blue solution. Following a 4 hour incubation period at 37 degrees C., samples were collected, plated in a fluorescence measurement system with 544 nm excitation and 590 nm emission. Control wells containing 10% Alamar blue solution were used to provide the background level measurements for oxidation of Alamar blue. Absorbance values were converted into cell numbers extrapolated from established standard curves. After 1 day the PFS modified sample according to the invention exhibited a remarkable acceleration of cell growth on its surface, 14,400 (±2,500) cells vs. 10,400 (±1,000) cells on the control sample. Samples taken after 4 days and 7 days also showed a remarkable acceleration of cell growth on the sample prepared according to the invention.

### EXAMPLE 2

In one example, a liquid bath was made from a mixture of 69 grams of distilled water, 11 grams of HNO3, 20 grams of tricalcium phosphate, and 8 grams of phosphoric acid (H3PO4). A sample disc of Ti-6Al-4V was submerged in the bath, grounded to the PFS circuit, and supported by a non-conductive polymeric support. A stream of argon was bubbled into the bottom of the bath for agitation. A PFS electrode of the same alloy was connected to the PFS apparatus, and placed in operative proximity to the sample. A relatively low energy PFS process was then conducted for about 3 minutes during which current was passed through the electrode and into the sample. The sample was then removed from the bath, ultrasonically cleaned, and analyzed by Energy-Dispersive X-Ray Spectroscopy (EDX) for calcium and phosphorous content. The PFS-applied layer included 7.33 atomic % calcium and 5.22 atomic % phosphorous. The sample was then tested for tissue-growth enhancement by the same methods as in Example 1.

Following a 1, 4 and 7 day culture period, attachment and proliferation was measured as was done in Example 1. After 1 day the PFS modified sample according to this embodiment of the invention exhibited a similar acceleration of cell growth on its surface, 14,500 (±1,900) cells vs. 10,400 (±1.000) cells on the control sample. Samples taken after 4 days and 7 days also showed a dramatic acceleration of cell growth on the sample prepared according to this embodiment of the invention.

It is believed that further development will reveal processing solutions and methods that provide even greater increases in cell growth and attachment rates. Accordingly, while the invention has been illustrated by way of the foregoing examples, it is not intended to be limited by those examples to the compositions or processing conditions therein. Those of skill in the art will understand that the methods and implants illustrated by way of the foregoing examples could be modified in numerous ways without departing from the scope of the invention.

1. A biocompatible structure comprising:
a body having a first surface;
a coating formed on the first surface and comprising at least one tissue-growth enhancing material;
a metallurgical bond connecting the coating to the first surface.

2. A biocompatible structure according to claim 1 wherein the coating is formed on the first surface by a submerged-arc welding process comprising the steps of:
submerging an electrode and the biocompatible structure in a liquid bath comprising the at least one tissue-growth enhancing material;
discharging a series of small electrical currents through the electrode and metallurgically bonding electrode material to the first surface thereby forming a coating comprising the electrode material and the at least one tissue growth enhancing material.

3. An implant according to claim 1 in which the submerged-arc welding process includes the step of discharging a series of small, controlled electrical currents through the electrode into the implant.

4. An implant according to claim 1 wherein the tissue-growth enhancing material comprises calcium.

5. An implant according to claim 1 wherein the tissue-growth enhancing material comprises calcium.

6. An implant according to claim 1 wherein the tissue-growth enhancing material comprises calcium and phosphorous.

7. An implant according to claim 2 wherein the liquid bath comprises dissolved calcium.

8. An implant according to claim 2 wherein the liquid bath comprises dissolved phosphorus.

9. An implant according to claim 2 wherein the liquid bath comprises dissolved calcium and dissolved phosphorus.

10. An implant according to claim 2 wherein the liquid bath comprises a finely divided solid comprising calcium.

11. An implant according to claim 2 wherein the liquid bath comprises a finely divided solid comprising phosphorus.

12. An implant according to claim 2 wherein the liquid bath comprises a finely divided solid comprising calcium and dissolved phosphorus.

13. An implant according to claim 1 wherein the coating formed on the first surface and comprising at least one tissue-growth enhancing material includes at least 0.05 atomic % of the at least one tissue-growth enhancing material.

14. An implant according to claim 1 wherein the coating formed on the first surface and comprising at least one tissue-growth enhancing material includes at least 0.5 atomic % of the at least one tissue-growth enhancing material.

15. An implant according to claim 1 wherein the coating formed on the first surface and comprising at least one tissue-growth enhancing material includes at least 1.0 atomic % of the at least one tissue-growth enhancing material.

16. An implant according to claim 1 wherein the coating formed on the first surface and comprising at least one tissue-growth enhancing material includes at least 5 atomic % of the at least one tissue-growth enhancing material.

17. An implant according to claim 1 wherein the implant comprises an orthopedic implant.

18. An implant according to claim 1 wherein the implant comprises a dental implant.

19. An implant according to claim 1 wherein the implant comprises a vascular implant.

20. An implant according to claim 1 wherein the implant comprises an implant attachment device.

21. An implant according to claim 1 wherein the implant is selected from the group consisting of hip and knee implants, spinal inserts, orthopedic and dental attachment devices such as screws and wires, cardiac devices, and vascular implants such as vascular occlusive devices.

22. An implant according to claim 2 wherein the step of forming the wear-resistant layer comprises depositing a layer of wear-resistant material by a pulsed fusion deposition process comprising the steps of:
providing an electrode comprising the wear-resistant material extending about a longitudinal axis;
connecting the electrode to an electrical current source;
positioning the electrode adjacent the first surface of the implant;
oscillating the electrode back and forth in a semi-circle about the longitudinal axis at a predetermined rate and in a predetermined pattern;
rotating the electrode completely about the longitudinal axis at the same time that the electrode is oscillating superimposing a 360 degree rotation into the predetermined semi-circular pattern; and
discharging a series of short-duration electrical current pulses from the current source through the electrode to the substrate, thereby melting and fusing a thin layer of the wear-resistant material and the tissue-growth enhancing material into the substrate.
A biocompatible structure according to claim 1 wherein the coating is formed on the first surface by a submerged-arc welding process comprising the steps of:

23. A method of forming a biocompatible structure comprising the steps of:
providing a substrate having a first surface;
providing an electrode comprising the wear-resistant material extending about a longitudinal axis;
connecting the electrode to an electrical current source;
submerging the electrode and the substrate in a liquid bath comprising the at least one tissue-growth enhancing material;
positioning the electrode adjacent the first surface of the substrate;
oscillating the electrode back and forth in a semi-circle about the longitudinal axis at a predetermined rate and in a predetermined pattern;
rotating the electrode completely about the longitudinal axis at the same time that the electrode is oscillating superimposing a 360 degree rotation into the predetermined semi-circular pattern; and
discharging a series of short-duration electrical current pulses from the current source through the electrode to the substrate, thereby melting and fusing a thin layer of the electrode material and the tissue-growth enhancing material into the first surface.
